(19) United States
(12) Patent Application Publication
(10) Pub. No.: US 2015/0329441 A1
(43) Pub. Date: Nov. 19, 2015

(54) Operation of Modified HF Alkylation Unit

(71) Applicants: Jeffrey M. Fitt, Centerville, VA (US); Stacey E. Johnson, Manassas, VA (US); Clay R. Sutton, Alexandria, VA (US); John J. Monson, League City, TX (US)

(72) Inventors: Jeffrey M. Fitt, Centerville, VA (US); Stacey E. Johnson, Manassas, VA (US); Clay R. Sutton, Alexandria, VA (US); John J. Monson, League City, TX (US)

(73) Assignee: EXXONMOBIL RESEARCH AND ENGINEERING COMPANY, Annandale, NJ (US)

(21) Appl. No.: 14/277,872

(22) Filed: May 15, 2014

(51) Int. Cl. C07C 2/60; C10L 1/16; C10L 10/10

(52) U.S. Cl. CPC C07C 2/60; C10L 10/10; C10L 1/1608

(57) ABSTRACT

An HF alkylation process for producing gasoline boiling range alkylate product by the alkylation in an HF alkylation unit of a light olefin reactant with an isoparaffin reactant in the presence of a hydrogen fluoride/sulfolane alkylation catalyst mixture in which fresh sulfolane feed having a Total Acid Number (TAN, ASTM D974) not more than 2 mq./L and optimally not more than 1 meq./L. is added to the hydrogen fluoride/sulfolane alkylation catalyst circulating in the unit. Control over the acidic components of the sulfolane feed is appropriately maintained by monitoring and pretreatment with an ion exchange resin to remove acidic components from the feed.

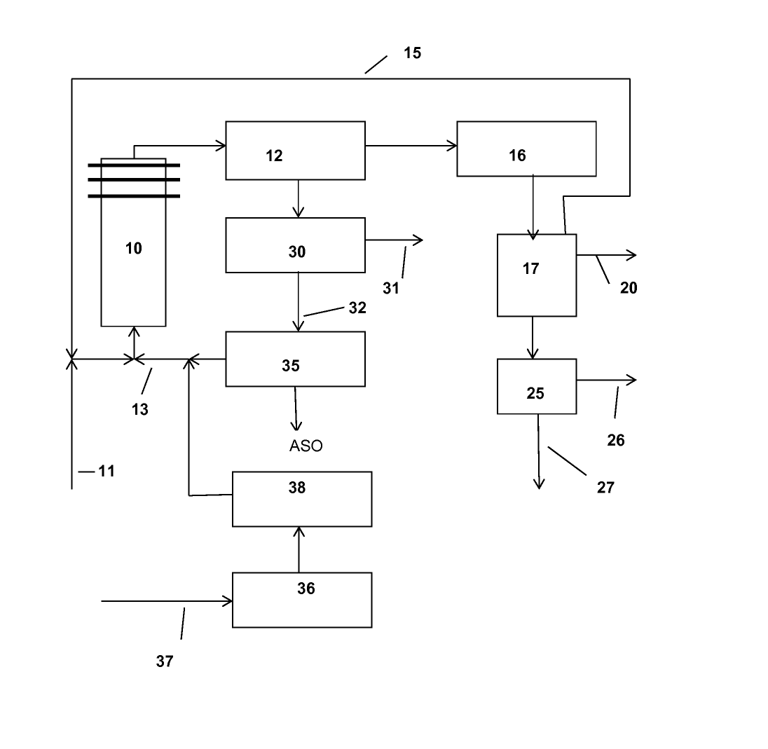

### FIELD OF THE INVENTION

The present invention relates to the isoparaffin: olefin alkylation process.

### BACKGROUND OF THE INVENTION

Alkylation is a reaction in which an alkyl group is added to an organic molecule. In the isoparaffin-olefin alkylation reaction, a low molecular weight isoparaffin is reacted with an olefin to provide an isoparaffin of higher molecular weight. Industrially, the concept depends on the reaction of a C₂ to C₅ olefin with a C₃ to C₆ olefin, typically isobutene, in the presence of an acidic catalyst producing a so-called alkylate. This alkylate is a valuable blending component in the manufacture of gasolines due not only to its high octane rating but also to its sensitivity to octane-enhancing additives; alkylate with its high octane rating and non-aromatic composition is particularly attractive as a blend component for reformulated gasoline and worldwide demand for alkylate as a component of the gasoline blend pool is expected to continue and grow.

Industrial alkylation processes have historically used concentrated hydrofluoric or sulfuric acid catalysts under relatively low temperature conditions. While both processes have achieved broad commercial success, the HF process, described in general terms in Modern Petroleum Technology, Hobson et al (Ed), Applied Science Publishers Ltd. 1973, ISBN 085334 487 6, offers a number of advantages and although hydrogen fluoride, or hydrofluoric acid (HF) is highly toxic and corrosive, extensive experience in its use in the refinery have shown that it can be handled safely, provided the hazards are recognized and precautions taken. The risks of inadvertent HF releases have also prompted mitigating efforts for some time. One approach that has achieved favor is the Modified HF Process in which sulfolane (tetramethylene sulfone) is added to the HF alkylation acid to reduce the evaporation of the HF in the event of an HF release into the atmosphere. The Modified HF Alkylation Process referred to as ReVap (Reduced Volatility Alkylation Process) was originally developed by Mobil Oil Corporation and Phillips Petroleum and is now offered by UOP. Features of the ReVap process are described in the paper by Shoemaker et al, HF and Reduced volatility HF Alkylation: Meeting the Challenge of the 21^{st} Century, Paper 29246, 15^{th} World Petroleum Congress, October 1997, Beijing. The process is also referred to in various patent publications including U.S. Pat. No. 5,191,150; and U.S. Pat. No. 5,264,651. A survey of HF alkylation may be found in Handbook of Petroleum Refining Processes, Meyers, R. A. (Ed.), McGraw-Hill Professional Publishing, 2nd edition (Aug. 1, 1996), ISBN 0070417962.

It has, however, been found that while concentrated HF is not particularly corrosive to mild steel, the mixture of HF and sulfolane is corrosive toward the grades of carbon steel which are commonly used for the construction of HF alkylation process units. Corrosion has tended to be most noted in the acid settler or decanter vessel which is one of the larger vessels present in the unit. The corrosivity problem is critical because the cost of retrofitting an existing HF alkylation process unit with corrosion-resistant alloy equipment could preclude replacing the concentrated HF catalyst with the safer HF/additive mixture.

Sulfolane itself is not considered corrosive to steel; corrosion of steel from using sulfolane generally occurs from by-products of sulfolane decomposition. Reactions with impurities present in the feed from upstream processes, reactions with oxygen in air, and high temperatures are known to cause sulfolane decomposition. The reaction of sulfolane with oxygen dissolved in the hydrocarbon feed, for example, creates sulfuric and sulfonic acids as well as other organic acids, aldehydes and ketones. Thermal decomposition occurs in sulfolane at higher temperatures and temperature also aggravates the reactions of sulfolane with oxygen. Corrosion problems in aromatic extraction units using sulfolane as the solvent has shown that reboiler regenerators and their associated equipment are common focal points for corrosion particularly in the presence of oxygen and while the actual alkylation step is carried out under relatively low temperature conditions, higher temperatures likely to create sulfolane degradation products are likely encountered in the various recovery sections.

The decomposition of sulfolane in the context of its uses in aromatics extraction of petroleum fractions and in the natural gas Sulfinol™ process has been discussed in the literature. In the Sulfolane Technical Assistance and Evaluation Report prepared for the Alaska Department of Environmental Conservation by Oasis Environmental, June 2010, the role of oxygen and chlorides as well as of thermally-induced degradation is recognized. Degradation products include sulfuric/sulfonic acids, as well as other organic acids, aldehydes and ketones. Oxygen-degraded sulfolane has a lower pH, a higher acid number, and a darker color than pure sulfolane. The patent literature has also presented proposals for removing the acid decomposition products. U.S. Pat. No. 7,288,184 (van Nuland) discloses a method for removing acids from the extraction solvent using a mixed cation/anion exchange resin; U.S. Pat. No. 4,820,849 (Diaz) discloses a process for reducing the amount of corrosive impurities in a sulfolane solution from an aromatics extraction process using a polyprotic acid to form a solid phase which is then removed from the sulfolane. U.S. Pat. No. 3,953,324 (Deal) uses a solid adsorbent to regenerate sulfolane from an extraction process and then regenerates the adsorbent by displacement with feed for the extraction process.

Currently, modified HF alkylation units mitigate the corrosion and operability issues by: monitoring the corrosion of equipment and replacing as necessary; removing the contaminated solvent via “dumping” a large quantity of the solvent/acid mixture to waste; lowering the unit feed rate to ensure more reliable operation; and removing the sulfolane from the system and operating in the more traditional HF alkylation configuration.

The first three alternatives are costly either in terms of capital or operating expenditure while the fourth is less inherently safe.

WO 93/19025 (Child) proposes to use a mixture of hydrofluoric acid, sulfolane, and water as an effective isoparaffin:olefin alkylation catalyst which exhibits a reduced tendency to form vapor clouds upon release and is stated to be less corrosive than an anhydrous mixture of HF and sulfolane. The addition of water, however, even in amounts of 1 to 5 percent of the acid/sulfolane inventory brings with it the disadvantage of increased corrosion to the base metal in both the reactor and fractionation sections. Accordingly, the need for an alternative corrosion mitigation strategy remains.

### SUMMARY OF THE INVENTION

We have now devised a new method of combatting the corrosion problems encountered in HF/sulfolane alkylation units. According to the present invention, an ion exchange resin is used to remove acidic components including sulfuric acid, sulfonic acids and other organic acids from the make-up sulfolane which is added to the unit. The acidity of the sulfolane should be maintained at a value of not more than 2 mq./L TAN (Total Acid Number) and optimally not more than 1 meq./L. and appropriate monitoring of the fresh sulfolane feed to the unit is suitably used to this end.

According to the present invention therefore, an HF alkylation process for producing gasoline boiling range alkylate product is carried out by alkylating a light olefin reactant with an isoparaffin reactant in the presence of a circulating hydrogen fluoride/sulfolane alkylation catalyst in an HF alkylation unit in which the fresh sulfolane feed added to the circulating HF/sulfolane catalyst mixture is controlled to have a Total Acid Number (TAN, ASTM D974) not more than 2 mq./L and optimally not more than 1 meq./L.. Control over the acidic components of the sulfolane feed is appropriately maintained by monitoring and pretreatment with an ion exchange resin to remove acidic components from the feed.

### DRAWINGS

The single FIGURE of the accompanying drawings is a simplified unit schematic of a modified HF alkylation unit using ion exchange resin to remove corrosive components from the make-up sulfolane.

### DETAILED DESCRIPTION

### Hydrocarbon Feedstocks

Hydrocarbon feedstocks useful in the present alkylation process include at least one C₄-C₈ isoparaffin and at least one C₂-C₁₂ olefin. Representative isoparaffins include isobutane, isopentane, 3-methylhexane, 2-r′ethylhexane, 2,3-dimethylbutane and 2,4-dimethylhexane with isobutene being the most common. Representative olefins include butene-2, isobutylene, butene-1, propylene, ethylene, hexene, octene, pentene, and heptene. The preferred olefins include the C₄ olefins, for example, butene-1. butene-2, isobutylene, or a mixture of one or more of these C₄ olefins, with butene-2 being the most preferred. Suitable feedstocks, for the process of the present invention are described in U.S. Pat. No. 3,862,258 to Huang et al. at column 3, lines 44-56. The molar ratio of isoparaffin to olefin is generally from 1:1 to 50:1, and more preferably from 5:1 to 20:1 with excess isoparaffin not consumed in the alkylation being internally recycled.

### Catalyst Composition

The catalyst typically comprises from 50 to 80 weight percent hydrofluoric acid together with from 20 to 50 weight percent sulfolane or another sulfone as described below and preferably from 20 to 40 weight percent sulfone and from 60 to 80 weight percent HF. The most preferred catalyst composition comprises from 25 to 35 weight percent sulfone together with from 65 to 75 weight percent HF. Controlling the water content within the range of from 0.5 to 10 weight percent, preferably from 1 to 5 weight percent, of the catalyst composition decreases the corrosivity of the catalyst toward carbon steel.

While sulfolane itself is the practical choice of vapor suppressing additive, other sulfones may also be used. Useful sulfones include those of the general formula:

R—SO₂—R¹

R and R¹ are monovalent hydrocarbon alkyl or aryl substituents, each containing from 1 to 8 carbon atoms, and wherein R and R¹ can be the same or different or together form an alicyclic ring as in the case of sulfolane itself (tetramethylene sulfone). Examples of suitable sulfones include, but are not limited to, dimethylsulfone, di-n-propylsulfone, diphenylsulfone, ethylmethylsulfone and alicyclic sulfones where the SO₂ group is bonded to a hydrocarbon ring. In such a case, R and R¹ are forming together a branched or unbranched hydrocarbon divalent moiety preferably containing from 3 to 12 carbon atoms. Among the latter, tetramethylenesulfone or sulfolane, 3-methylsulfolane and 2,4-dimethylsulfolane are more suitable since they offer the advantage of being liquid at the normal process operating conditions. These sulfones may also have substituents, particularly one or more halogen atoms, such as for example, chloromethylethylsulfone. These sulfones may be used in the form of mixtures.

### Process Unit

An exemplary HF-sulfolane olefin:isoparaffin alkylation unit is shown in the accompanying FIGURE. Other unit configurations are possible as are variant processes using a sulfone vapor pressure suppressor. The unit comprises a reactor 10 connected to a settling tank 11 in the conventional manner. Fresh hydrocarbon feed comprising olefin and isoparaffin enters the unit by way of line 12 and passes into the reactor along with recycled HF acid mixture from line 13 and recycle isobutene from line 15. The low density hydrocarbon phase is taken from the upper region of settling tank 11 to recontactor 16 (optional) and after passing through the recontactor passes to product separator 17 from which propane and butane are withdrawn by way of line 20 with the recycle isobutene passing out through line 15 back to the reactor. A bottoms comprising alkylate with residual water and sulfone additive passes to additive separator 25 from which alkylate product is taken by way of line 26 with separated water and additive components passing to the waste water treatment unit (not shown) by way of line 27.

The denser aqueous phase from the lower region of settler 11 passes to acid regeneration unit 30 from which water is withdrawn through line 31 while recycle acid mixture comprising HF acid, acid soluble oil (ASO), sulfone additive and water passes out through line 32 to additive recovery unit 35 which separates the undesirable acid soluble oil and returns alkylation acid mixture (HF, sulfone, water) to line 13 to enter the reactor as needed. Make up sulfone additive enters ion exchanger 36 from line 37 for removal of acidic impurities and then passes to additive injection pump 35 for feeding to the reactor by way of line 13.

The use of the recontactor is optional but preferred. If the recontactor is omitted from the alkylation unit, the light hydrocarbon phase will be taken directly from the settler to the product recovery section including the isostripper in which the isobutane is evaporated for return to the reactor and the hydrocarbons including propane and n-butane and alkylate product separated from the ASO; HF acid will be returned to the reactor from the HF rerun unit. The recontactor may suitably be of the type described in U.S. 2008/0139858 or U.S. 2009/0221863 using an eductor/mixer or microcontactor for the recontacting of the hydrocarbon with additional HF acid and optionally a hydrocyclone to promote separation of the hydrocarbon from the acid following the recontact between the hydrocarbon and acid phases. Separation of the hydrocarbons from the HF acid and the ASO may be carried out in various unit configurations since the use of the resin treatment to remove the acidic components from the sulfolane feed is an independent measure; accordingly, existing alkylation units may be used in combination with the resin treatment step without modification to the remaining sections of the unit although their operation, particularly the separation of the ASO and HF acid mixture from the alkylate and light ends will be facilitated and improved by the use of the resin treatment. Variations in reactor, settler and other sections of the unit will be apparent to those skilled in alkylation unit design and operation.

### Sulfolane Acidity and Monitoring

It is desirable to monitor the sulfolane feed to ensure that no acid molecules can enter the process and impede its reliability/operability. First, the sulfolane is treated on the resin(s) to remove the acidic components and second, the residual amounts of acids present either as free acids or salts are measured. Regular monitoring of the acid concentration is recommended. The Total Acid Number (TAN—ASTM D974) measurement is being applied to HF alkylation here for the first time. The sulfolane feed to the unit should not have a TAN>1 meq/L due to increased probability of corrosion. At TAN>2 meq/L it is expected that phase separation issues in the recontactor/decanter will become evident.

### Resin Treatment

The acidity (TAN) of the added sulfolane feed is suitably maintained within the specified limits by the use of a pre-treatment prior to the sulfolane entering the main portion of the unit, i.e. prior to entering the circulating reaction mixture. The objective of the resin treatment is to remove acidic contaminants and components from the make-up sulfolane before it enters the reactor. To this end, a mixed bed ion exchange bed treatment is preferred in order to remove both the acidic anions and the bases associated with them. Treatment with either type of ion exchanger will, however, effect an improvement although of a more limited character since removal of at least some of the corrosion-inducing components will take place. The preferred ion exchange resins for treatment of the sulfolane feed are macroreticular (i.e., large pore size) beads, however other resins, e.g., gel beads, may also be used. Macroreticular resins typically comprise a tough, rigid, spongelike material with large, discrete pores, and have a somewhat lower capacity than gel-type resins, but can have longer operating life and effect separations not possible with gel-type resins. High-molecular-weight ions can be more completely removed from solution and more completely eluted from the resin on regeneration. Moreover, the open structure allows application with tough copolymers which would be too dense for use in a strictly gel-type system. The backbones of the resins can be polystyrene, divinylbenzene, acrylic, or other polymers; a styrene-divinylbenzene backbone is preferred as resins of this type are readily available commercially and work well.

The anion exchange resin is preferably a strongly basic exchange resin since those resins have higher operating capacities, which means that the resin may be regenerated/replaced less frequently. Examples of strongly basic exchange resins include resins containing a quaternary ammonium group attached to a polymeric backbone with hydroxyl as the preferred counter ion so that when the amine function takes up the acid anions (e.g. chloride), it releases hydroxyl ions which combine with hydrogen ions released from the cation exchange resin to form water. The quaternary ammonium function is bonded to an organic polymer backbone, either directly or through one or more carbon atoms. The quaternary ammonium function may, for example, be the trimethylammonium hydroxide base, or the trimethylammonium chloride or trimethylammonium bromide groups bonded to a polystyrene backbone, that may be cross-linked with divinylbenzene. Examples of such strong anion exchange resins include the commercially available resins marketed by under the trademarks Amberlyst A-26, Amberlyst A-27, Amberlite IRA-904, Amberlite IRA-410, Amberlite IRA-400(OH), Amberlite IRA 458 and the resin marketed under the trademark Dowex 1X2-100.

The anion exchange resins may also be a macroreticular or gel type resin with weakly functional groups, such as the tertiary amine functional group. In such resins the tertiary amine function is bonded to an organic polymer e.g. polystyrene, backbone directly, or through one or more carbon atoms, or through a combination of carbon and nitrogen bonds. The backbone may be cross-linked with divinylbenzene. The tertiary amine function, may, for example, be the N,N-dimethylamine group or the N,N-diethylamine group. Alternatively, the tertiary amine group may be bonded directly, or indirectly to an acrylic acid or methacrylic acid polymer that may also be cross-linked with divinylbenzene, or to a phenolic polymer. Examples of such resins include the commercially available resins marketed under the trademarks Amberlyst A-21, Amberlite IRA-68, Amberlite IR-45, Amberlite XE-236 and the resin marketed under the trademark Duolite S-761.

Similarly, the preferred cation exchange resin is a strongly acidic resin with a similar advantage. The strong cation exchange resins typically derive their functionality from sulfonic acid groups linked to the backbone as described above, with the capability of exchanging cations in solutions with the protons on the acidic groups. Weak cation exchange resins typically derive functionality from carboxylic acid groups.

The use of a mixed bed cation/anion exchange resin is preferred in view of its capability of effecting the most complete removal of both cations and anions from the incoming sulfolane feed stream. Mixed resin types are commonly available with the resins already in the hydrogen and hydroxyl form, ready for use, e.g. Amberlite™ IRN-150 from Dow. One or more beds of mixed bed exchanger may suitably be used.

Resin regeneration should be considered when large quantities of make-up sulfolane are required. If resin regeneration is required, the anion removal resin could be regenerated with dilute NaOH KOH, NH₄OH, Mg(OH)₂, Ca(OH)₂, Sr(OH)₂, or Ba(OH)₂; the cation removal resin can be regenerated with dilute HCl, H₂SO₄, HNO₃, acetic acid or oxalate acid. With separate anion and cation exchangers, two separate resin beds will be required. The regeneration could be accomplished online or offline. Mixed bed resins will normally be discarded when spent. The ion exchanger(s) may be housed in two or more parallel vessels so that one bed may be regenerated or replaced while the other is online treating the feed. It is recommended that a filter be installed downstream of the resin to ensure that resin beds do mot migrate into the process.

As an example of the ability of the ion-exchange resin to remove the acidic molecules, sulfolane used at a major refinery HF alkylation unit was tested for TAN prior to treatment with an ion exchange resin and post treatment with a mixed bed ion exchange resin. The resin used was a mixture of uniform particle size gelular polystyrene mixed strongly acidic cation and strongly acidic anion exchange resins in the H⁺/OH⁻ forms (Amberlite IRN-150 by Rohm and Haas). The results shown below indicate that this resin can be used to remove the TAN molecules of Sulfolane A (without water) to the recommended level <0.2 meq/L.

| | Sulfolane used | Make Up |
|---|---|---|
| | in HF mixture | Sulfolane |
| Untreated TAN (meq/L) | 0.631 | 2.195 |
| Post Treated TAN (meq/L) | <0.1 | <0.1 |

In HF alkylation units, make-up sulfolane is typically added two ways. In one way it is added as a mixture with the HF acid, typically in an 85/15 acid/sulfolane ratio without water. The second way it is added pure (without water) to the decanter (settler). Regardless of which way is used with the unit, it is recommended to monitor and treat the source of make-up sulfolane for maximum benefit.

1. An HF alkylation process for producing gasoline boiling range alkylate product by the alkylation in an HF alkylation unit of a light olefin reactant with an isoparaffin reactant in the presence of a hydrogen fluoride/sulfolane alkylation catalyst in which fresh sulfolane feed having a Total Acid Number (TAN) of not more than 2 meq/L is added to the hydrogen fluoride/sulfolane alkylation catalyst circulating in the alkylation unit

2. An HF alkylation process according to claim 1 in which the Total Acid Number (TAN) of the fresh sulfolane feed is not more than 1 meq/L

3. An HF alkylation process according to claim 1 in which the fresh sulfolane feed is and is subjected to pretreatment with an ion exchange resin to remove acidic components in the sulfolane feed and bring the Total Acid Number (TAN) of the fresh sulfolane feed to a value of not more than 2 meq/L.

4. An HF alkylation process according to claim 3 in which the fresh sulfolane feed is and is subjected to pretreatment with an ion exchange resin to remove acidic components in the sulfolane feed and bring the Total Acid Number (TAN) of the fresh sulfolane feed to a value of not more than 1 meq/L.

5. An HF alkylation process according to claim 3 in which the fresh sulfolane feed is and is subjected to pretreatment with an anion exchange resin.

6. An HF alkylation process according to claim 3 in which the fresh sulfolane feed is and is subjected to pretreatment with an anion exchange resin.

7. An HF alkylation process according to claim 3 in which the fresh sulfolane feed is and is subjected to pretreatment with an anion exchange resin and a cation exchange resin.

8. An HF alkylation process according to claim 7 in which the fresh sulfolane feed is and is subjected to pretreatment with a mixed bed anion/cation exchange resin.

9. An HF alkylation process according to claim 3 in which the fresh sulfolane feed is and is subjected to pretreatment with a gel type ion exchange resin.

10. An HF alkylation process according to claim 7 in which the fresh sulfolane feed is and is subjected to pretreatment with a mixed bed anion exchange resin in the hydroxyl form and the cation exchange resin in the hydrogen form.

11. An HF alkylation process for producing gasoline boiling range alkylate product which comprises alkylating a light olefin reactant with an isoparaffin reactant in the presence of a hydrogen fluoride/sulfolane alkylation catalyst circulating in an HF alkylation unit in which fresh sulfolane feed is subjected to pretreatment with an ion exchange resin to remove acidic components in the sulfolane feed being added to the hydrogen fluoride/sulfolane alkylation catalyst circulating in the alkylation unit.

12. An HF alkylation process according to claim 11 in which the fresh sulfolane feed is and is subjected to pretreatment with an ion exchange resin to remove acidic components in the sulfolane feed and bring the Total Acid Number (TAN) of the fresh sulfolane feed to a value of not more than 2 meq/L.

13. An HF alkylation process according to claim 12 in which the fresh sulfolane feed is and is subjected to pretreatment with an ion exchange resin to remove acidic components in the sulfolane feed and bring the Total Acid Number (TAN) of the fresh sulfolane feed to a value of not more than 1 meq/L.

14. An HF alkylation process according to claim 11 in which the fresh sulfolane feed is and is subjected to pretreatment with an anion exchange resin.

15. An HF alkylation process according to claim 11 in which the fresh sulfolane feed is and is subjected to pretreatment with an anion exchange resin.

16. An HF alkylation process according to claim 11 in which the fresh sulfolane feed is and is subjected to pretreatment with an anion exchange resin and a cation exchange resin.

17. An HF alkylation process according to claim 16 in which the fresh sulfolane feed is and is subjected to pretreatment with a mixed bed anion/cation exchange resin.

18. An HF alkylation process according to claim 11 in which the fresh sulfolane feed is and is subjected to pretreatment with a gel type ion exchange resin.

19. An HF alkylation process according to claim 11 in which the fresh sulfolane feed is and is subjected to pretreatment with a mixed bed anion exchange resin with the anion exchange resin in the hydroxyl form and the cation exchange resin in the hydrogen form.

20. An HF alkylation process for producing gasoline boiling range alkylate product which comprises:
passing a stream of sulfolane catalyst additive containing acidic components over an ion exchange resin to remove acidic components in the sulfolane feed;
feeding the treated sulfolane catalyst additive to hydrogen fluoride/sulfolane alkylation catalyst circulating in an alkylation unit; and
alkylating a light olefin reactant with an isoparaffin reactant in the presence of the hydrogen fluoride/sulfolane alkylation catalyst in the alkylation unit.

21. An HF alkylation process according to claim 20 in which the stream of sulfolane catalyst additive following passage over the ion exchange resin has a Total Acid Number (TAN) of not more than 2 meq/L.

22. An HF alkylation process according to claim 20 in which the stream of sulfolane catalyst additive following passage over the ion exchange resin has a Total Acid Number (TAN) of not more than 2 meq/L.

23. An HF alkylation process according to claim 20 in which the stream of sulfolane catalyst additive is passed over an anion exchange resin.

24. An HF alkylation process according to claim 20 in which the stream of sulfolane catalyst additive is passed over a mixed bed anion/cation exchange resin.

25. An HF alkylation process according to claim 24 in which the stream of sulfolane catalyst additive is passed over a mixed bed anion/cation exchange resin with the anion exchange resin in the hydroxyl form and the cation exchange resin in the hydrogen form.
